(19) United States
(12) Patent Application Publication
(10) Pub. No.: US 2018/0044258 A1
(43) Pub. Date: Feb. 15, 2018

(54) ODOR SAMPLE FOR EXPLOSIVES DETECTION DOGS, PROCESS FOR PRODUCING AN ODOR SAMPLE AND PROCESS FOR USING AN ODOR SAMPLE

(71) Applicant: DIEHL DEFENCE GMBH & CO. KG, UEBERLINGEN (DE)

(72) Inventors: ARNO HAHMA, HENFENFELD (DE); OLIVER PHAM-SCHOENWETTER, LAUF (DE)

(21) Appl. No.: 15/673,464

(22) Filed: Aug. 10, 2017

(30) Foreign Application Priority Data
Aug. 12, 2016 (DE) 10 2016 009 872.4

(51) Int. Cl. C06B 23/00; F41H 11/132; A01K 15/02; G01N 33/00; G01N 33/22; C06B 25/04; F42B 8/00

(52) U.S. Cl. CPC C06B 23/006; C06B 25/04; F41H 11/132; F42B 8/00; G01N 33/0006; G01N 33/227; A01K 15/02

(57) ABSTRACT

An odor sample for explosives detection dogs includes a solution of the explosive in an ionic liquid, wherein the explosive is a nonperoxidic explosive. A process for producing an odor sample for explosives detection dogs as well as a process for using an odor sample for explosives detection dogs are also provided.

### CROSS-REFERENCE TO RELATED APPLICATION

This application claims the priority, under 35 U.S.C. §119, of German Patent Application DE 10 2016 009 872.4, filed Aug. 12, 2016; the prior application is herewith incorporated by reference in its entirety.

### BACKGROUND OF THE INVENTION

### Field of the Invention

The invention relates to an odor sample for explosives detection dogs, which includes a solution of the explosive in an ionic liquid. The invention also relates to a process for producing an odor sample and a process for using an odor sample.

The use of a solution composed of a neutral ionic liquid with a detectable amount of a peroxidic explosive as a scent source for training explosives detection dogs is known from German Publication DE 10 2009 029 787 A1, corresponding to U.S. Pat. Nos. 8,603,270 and 8,765,481. The peroxidic explosive can, for example, be triacetone triperoxide (TATP) or hexamethylene triperoxide diamine (HMTD). Dissolution of the explosive in the ionic solvent gives a stable and easily handleable form of the respective explosive. The mechanical and thermal sensitivity of the explosive is significantly reduced by dissolution in the ionic liquid, so that the solution can thus be handled easily in conventional laboratories with customary equipment. The dissolution of the peroxidic explosive in combination with a reductively active component in the ionic liquid permanently deactivates the explosive and can be utilized for desensitization. Reductively active ionic liquids enable stabilizing degradation of the peroxidic explosives to occur.

### SUMMARY OF THE INVENTION

It is accordingly an object of the invention to provide an odor sample for explosives detection dogs, a process for producing an odor sample and a process for using an odor sample, which overcome the hereinafore-mentioned disadvantages of the heretofore-known samples and processes of this general type and which provide an alternative scent source for training explosives detection dogs.

With the foregoing and other objects in view there is provided, in accordance with the invention, an odor sample for explosives detection dogs, comprising a solution of the explosive in an ionic liquid, wherein the explosive is a nonperoxidic explosive. The inventors of the odor sample according to the invention have surprisingly found that nonperoxidic explosives, for example hexogen (RDX), octogen (HMX), nitropenta (PETN), tetryl or trinitrotoluene (TNT), can also be dissolved readily and even in high concentration in ionic liquids. The dissolution in the ionic liquid desensitizes the explosive completely to friction, impact, percussion, shock, fire and any other stress. The solution is not explosive and thus also does not come under a hazard class encompassing explosives but only under a hazard class encompassing flammable liquids, with ionic liquids not being flammable per se. The odor sample according to the invention thus does not come within the scope of the German explosives law. It can be transported and handled without problems as a chemical. This applies even when the nonperoxidic explosive has been dissolved in a relatively high concentration in the ionic liquid. The high safety of the odor sample of the invention is also aided by the fact that the explosive present therein can only be separated from the ionic liquid with a high technical effort, e.g. chromatography. The amount of explosive obtainable in this way is small. Misuse of the odor sample of the invention for isolating a relevant amount of explosive is virtually ruled out thereby.

The odor sample of the invention, when it is stored, for example, in a suitable container which can be closed by using a screw cap, can be used for the training of explosives detection dogs for at least one year. The ionic liquid itself has no intrinsic odor since the vapor pressure of ionic liquids is negligibly small. As a result, the explosives detection dogs can perceive the pure odor of the explosive.

The ionic liquid can be a lipophilic ionic liquid. This is particularly well-suited for dissolving nonpolar nonperoxidic explosives. A further advantage of the lipophilic ionic liquid is that it takes up comparatively little water from the surroundings. Water can reduce the shelf life of the odor sample of the invention, in particular in the case of a hydrolysis-sensitive nonperoxidic explosive. Absorbed water can result in decomposition, in particular hydrolytic decomposition, of the explosive and thus also a change in the odor of the odor sample. The lipophilic ionic liquid can contain lipophilic anions.

The properties of the ionic liquid are influenced both by the anions forming the ionic liquid and by the cations forming the ionic liquid. Selection of the combination of anions and cations thus makes it possible to adapt the ionic liquid to the dissolution behavior of the respective explosive.

An ionic liquid having a relatively low viscosity can be selected. This is particularly suitable for impregnation of an absorptive support material such as kieselguhr.

The anions present in the ionic liquid can be anions selected from a group consisting of tetrafluoroborates, triflimides, perfluoroalkylsulphates, alkylsulphonates, dicyandiamides, alkylsulphates, arylsulphonates, perfluoroalkylsulphonates, bis-perfluoroalkylsulphonimides, acetates, alkylcarboxylates, thiocyanates, isocyanates, isothiocyanates, thiosulphates, borates, borohydrides, phosphates, nitrates, perchlorates and halides, in particular iodides, bromides, chlorides and fluorides.

The ionic liquid can contain cations selected from a group consisting of N-alkyl-substituted nitrogen heterocycle ions, in particular N-alkylpyridinium, N-alkylimidazolium and N,N-dialkylimidazolium ions, quaternary ammonium ions and phosphonium ions. N,N-dialkylimidazolium and N-alkylpyridinium ions are particularly suitable.

The ionic liquid can be selected from a group consisting of:
1-ethyl-3-methyl-imidazolium ethylsulphate,
1-ethyl-3-methylimidazolium-bis(trifluoromethanesulphonimide),
1-butyl-3-methylimidazolium-bis(trifluoromethanesulphonimide),
1-hexyl-3-methylimidazoliumbis(trifluoro-methanesulphonimide),
1-ethyl-3-methylimidazolium thiocyanate,
1-butyl-3-methylimidazolium thiocyanate,
1-ethyl-3-methylimidazolium tetrafluoroborate,
N-(n-hexyl)pyridinium tetrafluoroborate,
N-(n-hexyl)pyridinium bis(trifluoromethanesulphonimide),
N-(n-butyl)-3-methylpyridinium tetrafluoroborate,
N-(n-butyl)-4-methylpyridinium tetrafluoroborate,
N-methylpyrrolidine-zinc borohydride,
1-allyl-3-n-butylimidazolium borohydride,
1,3-diallylimidazolium borohydride,
1,3-di(n-octyl)imidazolium borohydride and
1,3-di(n-butyl)imidazolium borohydride.

In one embodiment of the invention, the ionic liquid itself has a reducing action with respect to the explosive or the ionic liquid contains a reducing agent which reduces the explosive in the ionic liquid. Reduction of the explosive destroys the latter so that even a theoretically possible separation with great effort, for example by using chromatography, of the explosive from the ionic liquid can no longer give a functioning explosive. This makes the possibility of misuse of the odor sample of the invention for obtaining explosive virtually impossible. The reducing agent can, for example, be a sugar, a sulphite, dithionite, thiosulphate, hydrazine, borane, phosphine, a hydride, zinc, a siloxane or a silane. The sugar can be glucose, powdered sugar, fructose, galactose, maltose or lactose. The hydride can be a metal hydride, for example lithium aluminum hydride or borohydride.

The ionic liquid which itself has a reducing action with respect to the explosive can be a thiocyanate, in particular 1-ethyl-3-methylimidazolium thiocyanate or 1-butyl-3-methylimidazolium thiocyanate, or a borohydride, in particular N-methylpyrrolidine-zinc borohydride, 1-allyl-3-n-butylimidazolium borohydride, 1,3-diallylimidazolium borohydride, 1,3-di(n-octyl)imidazolium borohydride or 1,3-di(n-butyl)imidazolium borohydride.

A high degree of safety with respect to a risk of explosion is offered by an odor sample according to the invention in which the explosive is present in a concentration of not more than 20% by weight, in particular not more than 15% by weight, in particular not more than 12.5% by weight, in particular not more than 10% by weight, in the ionic liquid.

In order for the odor sample to keep for a very long time and for a very small volume thereof to be sufficient as an odor sample for explosives detection dogs, the explosive should be present in a concentration of at least 1% by weight, in particular at least 2.5% by weight, in particular at least 5% by weight, in particular at least 7.5% by weight, in particular at least 9% by weight, in the ionic liquid.

With the objects of the invention in view, there is also provided a process for producing the odor sample of the invention, wherein the nonperoxidic explosive is dissolved in the ionic liquid.

With the objects of the invention in view, there is concomitantly provided a process for using the odor sample of the invention as a scent source, in particular for test measurements for calibrating detectors and for the training of explosives detection dogs or of other animals suitable for detecting explosive, in particular rats.

Other features which are considered as characteristic for the invention are set forth in the appended claims.

Although the invention is illustrated and described herein as embodied in an odor sample for explosives detection dogs, a process for producing an odor sample and a process for using an odor sample, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a diagram showing the structural formulae of examples of ionic liquids;

FIG. 2 is a diagram showing a 1H-NMR spectrum of 1-ethyl-3-methylimidazolium ethylsulphate;

FIG. 3 is a diagram showing a 1H-NMR spectrum of TNT;

FIG. 4 is a diagram showing a 1H-NMR spectrum of 10% by weight of TNT in 1-ethyl-3-methyl-imidazolium ethylsulphate;

FIG. 5 is a diagram showing a headspace gas chromatography-mass spectrum of the gas space in an empty flask;

FIG. 6 is a diagram showing a headspace gas chromatography-mass spectrum of the gas space in a flask containing 1-ethyl-3-methylimidazolium ethylsulphate; and

FIG. 7 is a diagram showing a headspace gas chromatography-mass spectrum of the gas space in a flask containing 10% by weight of TNT in 1-ethyl-3-methylimidazolium ethylsulphate.

### DETAILED DESCRIPTION OF THE INVENTION

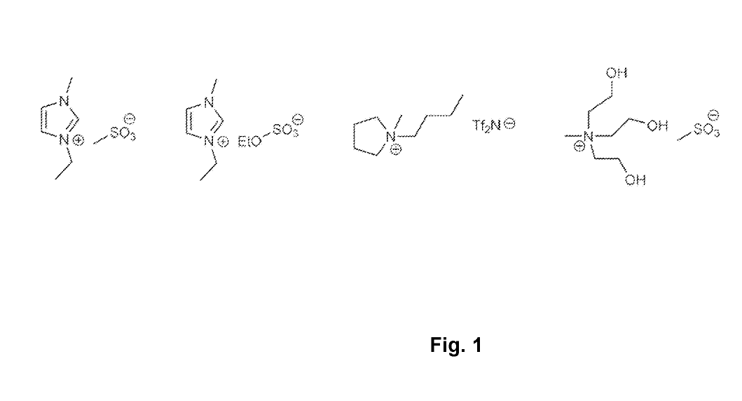

Referring now to the figures of the drawings in detail and first, particularly, to FIG. 1 thereof, there are seen structural formulae of examples of ionic liquids.

A 500 ml glass flask which is made of dark glass and can be closed tightly by using a closure having a Teflon seal is provided with a magnetic stirrer bar and charged with 270 g of 1-ethyl-3-methylimidazolium ethylsulphate. 30 g of TNT are added carefully a little at a time while stirring. The glass flask is closed by using the closure and stirred for at least 12 hours by using the magnetic stirrer bar and a magnetic stirrer at 350 revolutions per minute until the TNT has completely dissolved in the ionic liquid. The result is a solution containing 10% by weight of TNT.

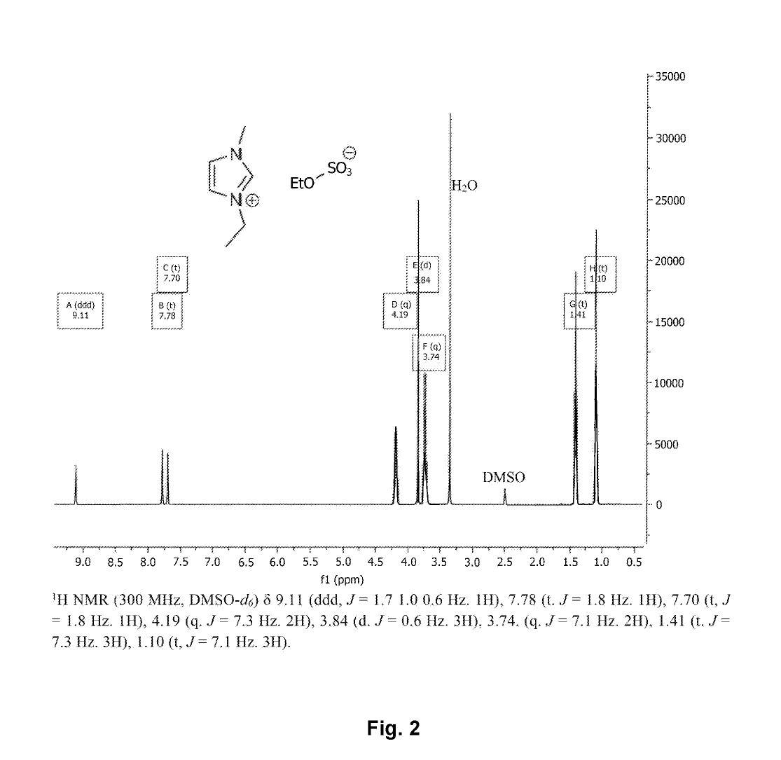

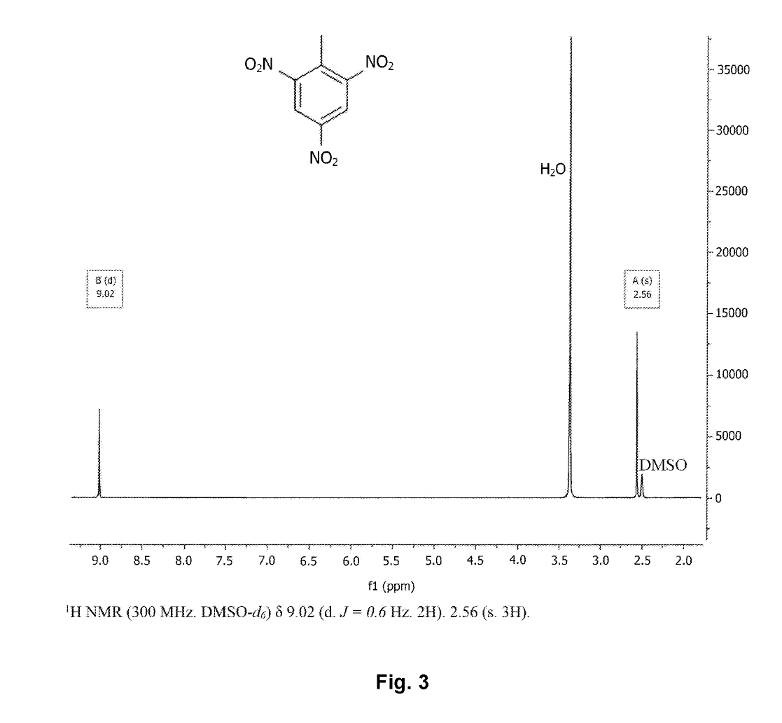

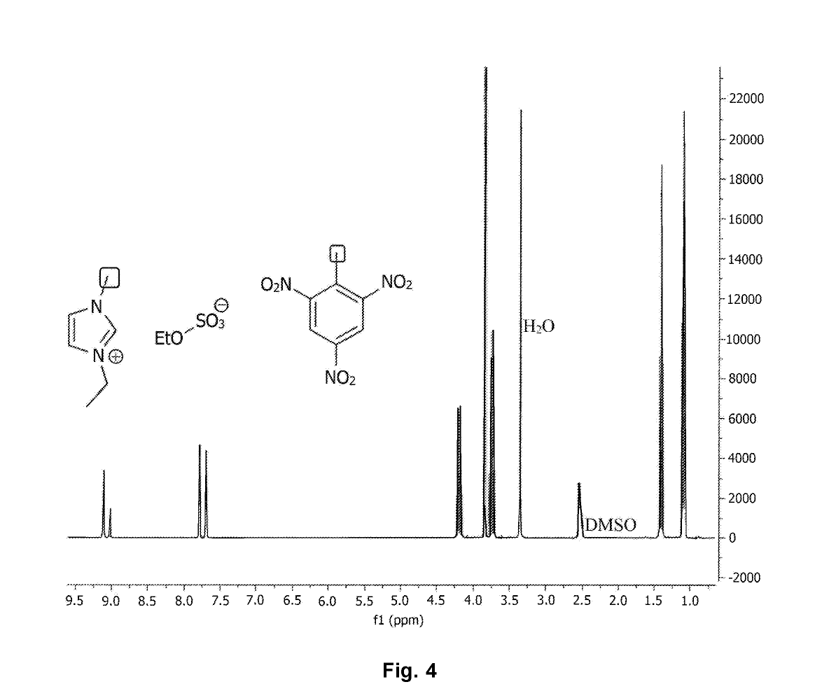

A comparison of the 1H-NMR spectra of 1-ethyl-3-methylimidazolium ethylsulphate according to FIG. 2, TNT according to FIG. 3 and the TNT solution prepared as described above according to FIG. 4 unambiguously shows the presence of TNT in the ionic liquid. The DMSO peak present in the spectra results from contamination of the deuterated DMSO used as solvent for the spectra with undeuterated DMSO.

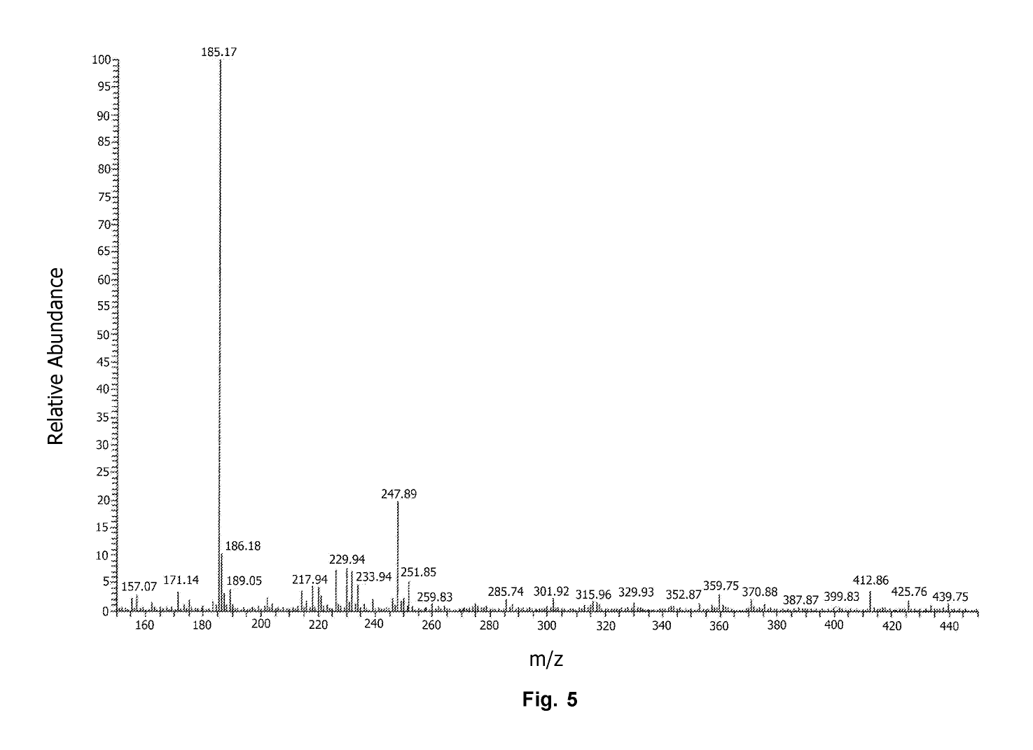

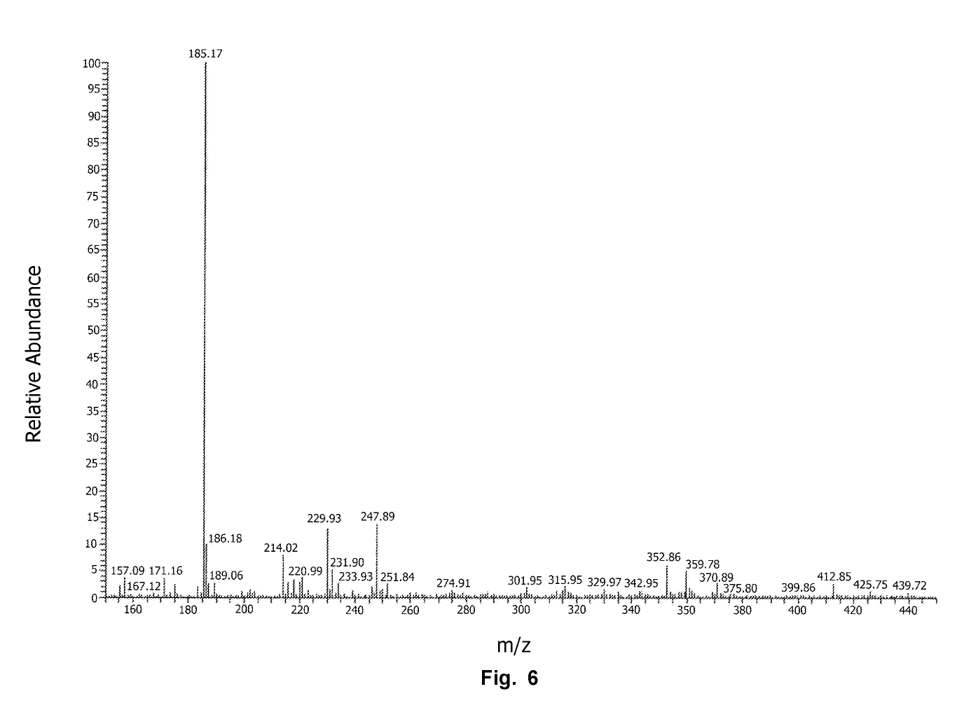

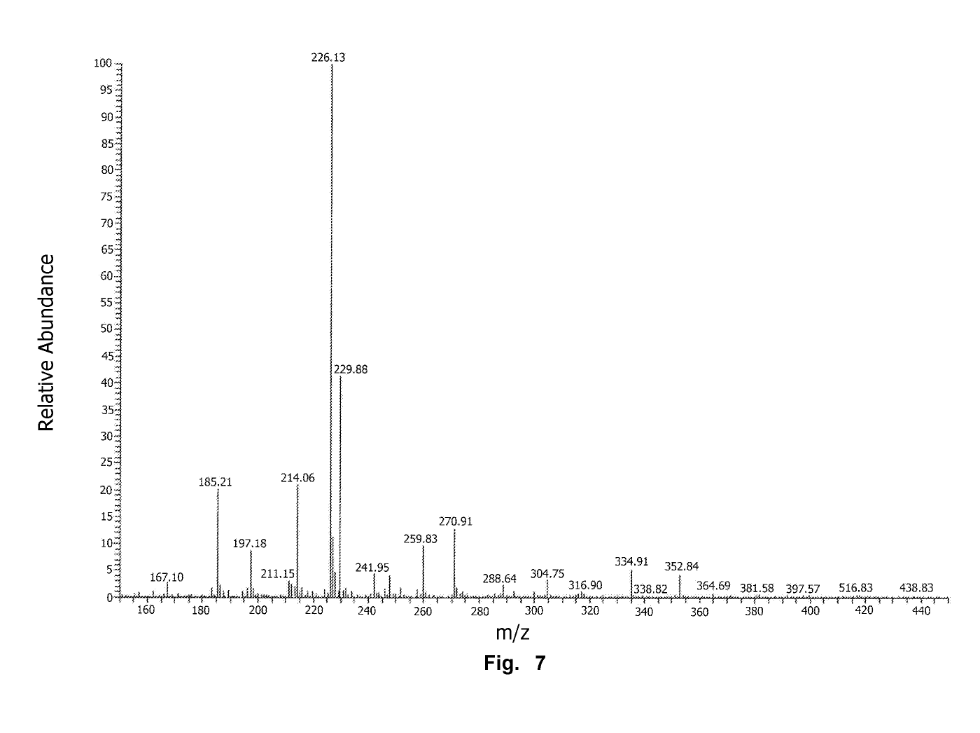

It can be seen from a comparison of FIG. 6 with FIG. 5 that the substances detectable in the mass spectrum of the gas phase over the ionic liquid do not differ from the substances which could be detected in the mass spectrum of the gas space in the empty flask. In contrast, the mass spectrum of the gas phase over the ionic liquid containing 10% by weight of TNT, according to FIG. 7, clearly shows the presence of TNT. This shows that the ionic liquid does not have any influence on the substances in the gas phase over the solution of the explosive in the ionic liquid and the explosive in this gas phase is detectable and thus can also be sniffed out by an explosives detection dog.

1. An odor sample for explosives detection dogs, the odor sample comprising:
a solution of an explosive in an ionic liquid;
said explosive being a nonperoxidic explosive.

2. The odor sample according to claim 1, wherein said nonperoxidic explosive is hexogen (RDX), octogen (HMX), nitropenta (PETN), tetryl or trinitrotoluene (TNT).

3. The odor sample according to claim 1, wherein said ionic liquid is a lipophilic ionic liquid.

4. The odor sample according to claim 3, wherein said ionic liquid contains lipophilic anions.

5. The odor sample according to claim 1, wherein said ionic liquid contains anions selected from the group consisting of tetrafluoroborates, triflimides, perfluoroalkylsulphates, alkylsulphonates, dicyandiamides, alkylsulphates, arylsulphonates, perfluoroalkylsulphonates, bis-perfluoroalkylsulphonimides, acetates, alkylcarboxylates, thiocyanates, isocyanates, isothiocyanates, thiosulphates, borohydrides, borates, phosphates, nitrates, perchlorates and halides.

6. The odor sample according to claim 5, wherein said ionic liquid contains iodides, bromides, chlorides or fluorides.

7. The odor sample according to claim 1, wherein said ionic liquid contains cations selected from the group consisting of N-alkyl-substituted nitrogen heterocycle ions, N-alkylimidazolium and N,N-dialkylimidazolium ions, quaternary ammonium ions and phosphonium ions.

8. The odor sample according to claim 7, wherein said N-alkyl-substituted nitrogen heterocycle ions are N-alkylpyridinium.

9. The odor sample according to claim 1, wherein said ionic liquid is selected from the group consisting of:
1-ethyl-3-methylimidazolium ethylsulphate,
1-ethyl-3-methylimidazoliumbis(trifluoromethanesulphonamide),
1-butyl-3-methylimidazoliumbis(trifluoromethanesulphonimide),
1-hexyl-3-methylimidazoliumbis(trifluoromethanesulphonimide),
1-ethyl-3-methylimidazolium thiocyanate,
1-butyl-3-methylimidazolium thiocyanate,
1-ethyl-3-methylimidazolium tetrafluoroborate,
N-(n-hexyl)pyridinium tetrafluoroborate,
N-(n-hexyl)pyridiniumbis(trifluoromethanesulphonimide),
N-(n-butyl)-3-methylpyridinium tetrafluoroborate,
N-(n-butyl)-4-methylpyridinium tetrafluoroborate,
N-methylpyrrolidine-zinc borohydride,
1-allyl-3-n-butylimidazolium borohydride,
1,3-diallylimidazolium borohydride,
1,3-di(n-octyl)imidazolium borohydride and
1,3-di(n-butyl)imidazolium borohydride.

10. The odor sample according to claim 1, wherein said ionic liquid itself has a reducing action relative to said explosive or contains a reducing agent reducing the explosive.

11. The odor sample according to claim 10, wherein said reducing agent is a sugar, a sulphite, dithionite, thiosulphate, hydrazine, borane, phosphine, a hydride, a metal hydride, zinc, magnesium, a siloxane or a silane.

12. The odor sample according to claim 11, wherein said sugar is glucose, fructose, galactose, maltose or lactose and said metal hydride is lithium aluminum hydride or borohydride.

13. The odor sample according to claim 10, wherein said ionic liquid which itself has a reducing action relative to the explosive is a thiocyanate or a borohydride.

14. The odor sample according to claim 13, wherein said thiocyanate is 1-ethyl-3-methylimidazolium thiocyanate or 1-butyl-3-methylimidazolium thiocyanate.

15. The odor sample according to claim 13, wherein said borohydride is N-methylpyrrolidine-zinc borohydride, 1-allyl-3-n-butylimidazolium borohydride, 1,3-diallylimidazolium borohydride, 1,3-di(n-octyl)imidazolium borohydride or 1,3-di(n-butyl)imidazolium borohydride.

16. The odor sample according to claim 1, wherein the explosive is present in a concentration of not more than 20% by weight, not more than 15% by weight, not more than 12.5% by weight or not more than 10% by weight in the ionic liquid.

17. The odor sample according to claim 1, wherein the explosive is present in a concentration of at least 1% by weight, at least 2.5% by weight, at least 5% by weight, at least 7.5% by weight or at least 9% by weight in the ionic liquid.

18. A process for producing an odor sample for explosives detection dogs, the process comprising dissolving a nonperoxidic explosive in an ionic liquid.

19. A process for using an odor sample, the process comprising using a solution of a nonperoxidic explosive in an ionic liquid as a scent source for explosives detection dogs.

20. The process according to claim 19, which further comprises using the scent source for test measurements for calibrating detectors.

21. A process for using an odor sample, the process comprising using a solution of a nonperoxidic explosive in an ionic liquid for training explosives detection dogs or other animals suitable for detecting explosive.

22. The process according to claim 21, wherein the other animals are rats.
